(19) United States
(12) Patent Application Publication
(10) Pub. No.: US 2012/0051576 A1
(43) Pub. Date: Mar. 1, 2012

(54) EAR PAD

(75) Inventors: Shunsuke Shiomi, Tokyo (JP); Hiroaki Ishiguro, Tokyo (JP); Nobuyuki Ishizaki, Tokyo (JP)

(73) Assignee: Sony Corporation, Tokyo (JP)

(21) Appl. No.: 13/216,705

(22) Filed: Aug. 24, 2011

(30) Foreign Application Priority Data
Sep. 1, 2010 (JP) 2010-196012

(51) Int. Cl. H04R 1/10; A61F 11/14

(52) U.S. Cl. 381/374; 2/410

(57) ABSTRACT

An ear pad for use with a headphone apparatus. The ear pad comprises a pad portion comprising an outer circumference and an inner circumference defining an opening. The pad portion comprises an upper width between the outer and inner circumferences along a vertical mid-line of the pad portion, a lower width between the outer and inner circumferences along the vertical mid-line of the pad portion, a left side width between the outer and inner circumferences along a horizontal mid-line of the pad portion, and a right side width between the outer and inner circumferences along the horizontal mid-line of the pad portion. The lower width of the pad portion is greater than at least one of the left and right side widths of the pad portion.

### BACKGROUND

The present disclosure relates to an ear pad, and more particularly to an ear pad which is provided in a headphone or the like as a buffer member.

In the related art, headphones for listening to sound output from a sound reproduction device have been used. The headphones typically include a headband coming into contact with the head of a user, and a housing containing a sound output means which is provided at both ends of the headband and which is constituted by speakers and the like. On the surface of the housing facing the temporal region of the user, an ear pad which prevents the housing from coming into direct contact with the temporal region of the user is provided as a buffer member.

The ear pad functions as a buffer member for reducing a sense of discomfort caused by headphones coming into contact with the ear, the temporal region or the like of the user when they are worn. In addition, the ear pad is constituted by a material having high air tightness and seals a gap with the temporal region of the user, thereby facilitating listening to sound from a speaker by blocking noise from the outside. The ear pad also prevents sound from the speaker from being leaked to the outside.

The ear pad has various shapes, and, for example, there is an ear pad in which an outer diameter has the same distance from an inner diameter. In addition, there are an ear pad which has a circular shape having a constant width, an ear pad which has a substantial rectangular shape having a constant width, and the like. However, in these shapes having a constant width, as shown in FIG. 1, hair around the ear is interposed between the ear pad 1000 and the temporal region 2000 of the user when the headphones are worn. Thereby, a space formed by the ear pad 1000 and the temporal region 2000 of the user communicates with the outside, which thus decreases the sealing performance. In addition, the sealing performance is decreased due to occurrence of a gap between the unevenness in the temporal region around the ear and the ear pad.

If the sealing performance is low, the ear pad fails to prevent sound from being leaked to the outside. Further, the bass of output sound is reproduced insufficiently. FIG. 2A, in which the transverse axis indicates the frequency, and the longitudinal axis indicates a sound pressure of sound to be reproduced, shows a comparison result of sound pressures between a headphone provided with an ear pad having a high degree of sealing and a headphone provided with an ear pad having a low degree of sealing. As can be seen from FIG. 2A, in the case of the low degree of sealing, the sound pressure is lowered in the region having a low frequency, that is, in the bass, and thus the bass is not sufficiently reproduced.

In addition, the penetration of noise from the outside causes a problem. FIG. 2B, in which the transverse axis indicates the frequency, and the longitudinal axis indicates a noise penetration level, shows a result in which noise penetration levels between a headphone provided with an ear pad having the high degree of sealing and a headphone provided with an ear pad having the low degree of sealing are compared. The comparison is made using an unsealed headphone, a headphone having the low degree of sealing, and a headphone having the high degree of sealing. As can be from FIG. 2B, the high degree of sealing can reduce the noise penetration level.

Therefore, increasing the sealing performance through an increase in lateral pressure of the headband constituting the headphones has been performed. Since the ear pad is deformed following the shape of the temporal region of the user when the user wears the headphones, increasing the sealing performance through increase in the contact area with the head of the user by forming the ear pad itself to be large has also been performed.

In addition, there has been proposed an ear pad which includes a ring shape buffer member and a covering member which covers the ring shape buffer member, in which the ring shape buffer member includes an outer ring member, an intermediate ring member, and an inner ring member, and the hardness of at least the intermediate ring member is different from that of the outer ring member and the inner ring member (Japanese Unexamined Patent Application Publication No. 2009-105841).

In the ear pad disclosed in Japanese Unexamined Patent Application Publication No. 2009-105841, since the hardness of the intermediate ring member is lower than that of the inner ring member and the outer ring member, if the ring shape buffer material is covered by the covering member, the outer ring member and the inner ring member are distorted toward the intermediate ring member by the tension of the covering member. As a result, the distortion absorbs the tension of the covering member, and thus the upper surfaces of the outer ring member and the inner ring member are substantially planarized. Since the head of a user comes into contact with the planarized surfaces, the contact area between the head of the user and the ear pad increases, and thus the sealing performance by the ear pad can be heightened.

### SUMMARY

However, in the method of increasing the lateral pressure of the headband, in other words, since a tightening force on the ears or the head of the user becomes large, the user has a sense of discomfort or feels pain. In addition, in the method of forming the ear pad to be large, the portability of the headphones is compromised.

It is possible to increase the sealing performance without compromising the portability of the headphones by the use of the ear pad disclosed in Japanese Unexamined Patent Application Publication No. 2009-105841. However, the sealing performance thereof may still not be sufficiently satisfactory. The sealing performance may be increased by giving a feature to the shape of the ear pad in addition to giving a feature to the material of the ear pad.

It is desirable to provide an ear pad capable of increasing the sealing performance, and suppressing leakage of sound to the outside, penetration of noise, and the like.

One embodiment described herein is directed to an ear pad for use with a headphone apparatus. The ear pad comprises a pad portion comprising an outer circumference and an inner circumference, the inner circumference defining an opening. The pad portion comprises an upper width between the outer and inner circumferences along a vertical mid-line of the pad portion, a lower width between the outer and inner circumferences along the vertical mid-line of the pad portion, a left side width between the outer and inner circumferences along a horizontal mid-line of the pad portion, and a right side width between the outer and inner circumferences along the horizontal mid-line of the pad portion. The lower width of the pad portion is greater than at least one of the left and right side widths of the pad portion.

Another embodiment described herein is directed to a headphone apparatus. The headphone apparatus comprises a headband comprising left and right ends, left and right housings respectively coupled to the left and right ends of the headband, and left and right ear pads respectively coupled to the left and right housings. Each of the left and right ear pads comprises a pad portion having an outer circumference and an inner circumference, the inner circumference defining an opening. Each pad portion comprises an upper width between the outer and inner circumferences along a vertical mid-line of the pad portion, a lower width between the outer and inner circumferences along the vertical mid-line of the pad portion, a left side width between the outer and inner circumferences along a horizontal mid-line of the pad portion, and a right side width between the outer and inner circumferences along the horizontal mid-line of the pad portion. The lower width of each pad portion is greater than at least one of the left and right side widths of the pad portion.

A further embodiment described herein is directed to a method of increasing the sealing performance of an ear pad comprising a pad portion. The pad portion comprises an upper width and a lower width along a vertical mid-line of the pad portion and a left side width and a right side width along a horizontal mid-line of the pad portion, wherein the lower width is greater than at least one of the left side width and the right side width. The method comprises positioning the pad portion so that a lower region of the pad portion including the lower width contacts a region under an ear of a user.

According to the present disclosure, it is possible to provide an ear pad capable of increasing the sealing performance, and suppressing leakage of sound to the outside, penetration of noise, and the like, without employing the method of increasing lateral pressure of a headband or the like.

### BRIEF DESCRIPTION OF THE DRAWINGS

FIG. 1 is a diagram illustrating problems in an ear pad in the related art.

FIG. 2A is a diagram illustrating a relationship between a degree of sealing of an ear pad and sound pressure.

FIG. 2B is a diagram illustrating a relationship between a degree of sealing of an ear pad and noise penetration level.

FIG. 3 is a diagram illustrating an exterior configuration of headphones provided with ear pads according to an embodiment of the present disclosure.

FIGS. 4A to 4E are exterior views of an ear pad according to a first embodiment.

FIGS. 5A and 5B are cross-sectional views of the ear pad according to the first embodiment.

FIGS. 6A to 6C are diagrams illustrating the temporal region of a user with which the ear pad comes into contact.

FIGS. 7A and 7B are diagrams illustrating relationships between the outer sizes of the ear pad in the vertical direction, a noise penetration level, and a noise blocking level.

FIGS. 8A to 8E are exterior views of an ear pad according to a second embodiment.

FIGS. 9A and 9B are cross-sectional views of the ear pad according to the second embodiment.

FIGS. 10A and 10B are diagrams illustrating states of the ear pad when headphones are worn.

FIGS. 11A and 11B are diagrams illustrating modified examples of the ear pad according to the embodiments of the present disclosure.

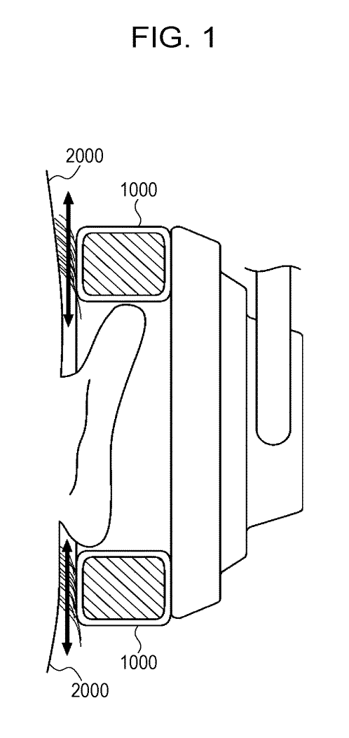

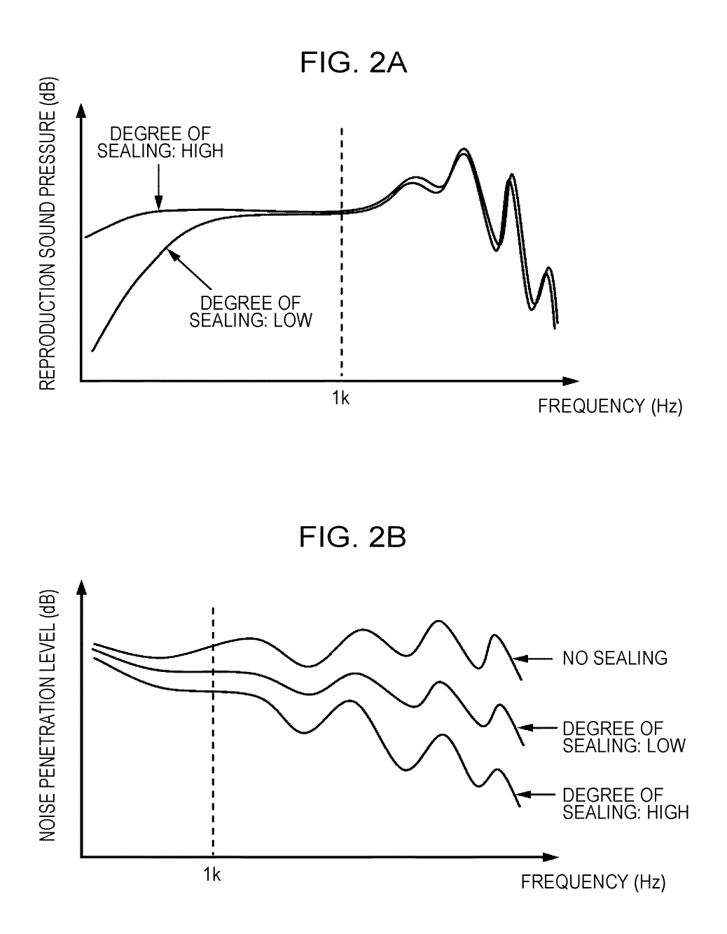

### DETAILED DESCRIPTION OF EMBODIMENTS

Hereinafter, embodiments of the present disclosure will be described with reference to the accompanying drawings. The description will be made in the following order.

1. First Embodiment

1.1. Configurations of Headphone and Ear Pad

1.2. Effects Achieved by Ear Pad

2. Second Embodiment

2.1. Configuration of Ear Pad

2.2. Effects Achieved by Ear Pad

3. Modified Examples

### 1. First Embodiment

### 1.1. Configurations of Headphone and Ear Pad

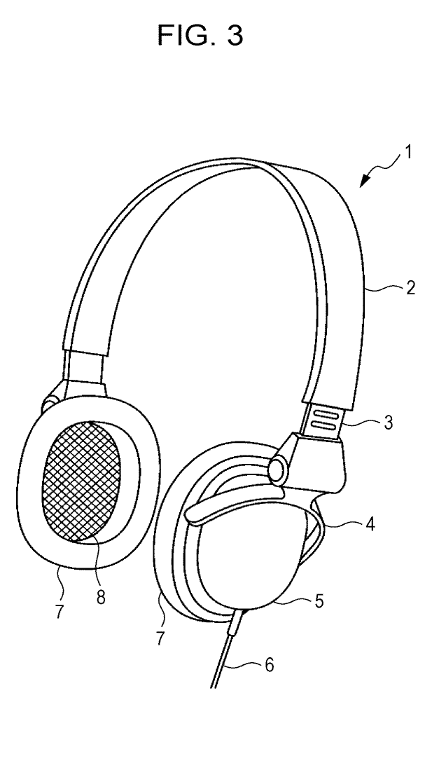

FIG. 3 is a diagram illustrating an exterior configuration of headphones 1 having ear pads 7 according to an embodiment of the present disclosure. The headphones 1 include a headband 2, sliders 3, hangers 4, housings 5, a cord 6, and the ear pads 7.

The headband 2 is formed in a bent shape so as to fit the head of a user, and supports the overall headphones 1 by coming into contact with the top of the head of the user in a state of wearing the headphones 1. The headband 2 is formed using synthetic resin such as plastic, metal, or the like, and is flexible due to predetermined stiffness and elasticity. Thereby, when the headphones 1 are worn, it is possible to maintain the state of wearing the headphones 1 by pressing the housings 5 and the ear pads 7 in the direction of the temporal region of the user. In addition, rubber or the like may be provided at a part which comes into contact with the top of the head of the user in the inner surface of the headband 2. A hinge may be provided so as to be folded at the center of the headband 2 when the headphones 1 are carried.

The sliders 3 are provided at both ends of the headband 2. The hangers 4 are provided at the sliders 3. The sliders 3 are configured to be slidable inside the headband 2. When the sliders 3 are slid, the hangers 4 can be moved upward or downward with respect to the headband 2. When the headphones 1 are worn, the housings 5 and the ear pads 7 can fit in positions facing the ears of the user by adjusting the extensible degree of the sliders 3 according to the size of the head of the user or the distance between the ears and the top of the head. Thereby, the user can obtain a sense of wear suitable for the physical features or preferences of the user. On the other hand, when the headphones 1 are not used, it is possible to save space for storing them by shortening the sliders 3.

The hangers 4 are provided at the front ends of the sliders 3 and support the housings 5 to be rotated. The hangers 4 support the housings 5 to be rotated by axially supporting the support pins (not shown), which respectively protrude from a pair of front ends to the inside. Therefore, when the headphones 1 are worn, since the directions of the housings 5 vary according to the shapes of the vicinities of the ears of the user, the housings 5 can face the ears in a state suitable for the shape of the temporal region of the user.

The housings 5 function as containers containing sound processing circuits, speakers (none of them are shown), and the like therein. The housings 5 are formed using, for example, a synthetic resin such as plastic. The sound processing circuit performs, for example, a predetermined sound signal process such as a noise cancelling process, a signal amplifying process, or an equalizing process for a sound signal which drives the speaker. The speaker is a sound output unit which outputs the sound signal which has been processed by the sound processing circuit, as a sound.

The cord 6 inserts a left channel conducting wire L, a right channel conducting wire R, a ground wire G, and the like therein, and transmits sound signals. One end of the cord 6 is connected to the sound processing circuit contained in one housing 5 of the pair of housings 5. A plug (not shown) is provided at the other end of the cord 6. The plug is connected to a sound reproduction device (not shown) such as an MP3 player and thus the headphones 1 are connected to the sound reproduction device.

In order to drive the speaker in the other housing 5, which is not connected to the cord 6, a connection cord (not shown) is provided between the one housing 5 connected to the cord 6 and the other housing 5 not connected to the cord 6. The connection cord is connected to the cord 6 or the sound processing circuit in the housing 5 connected to the cord 6, and is connected to the sound processing circuit in the other housing 5 via the insides of the hangers 4, the sliders 3, and the headband 2. Due to this connection cord, sound signals are transmitted to the sound processing circuit in the other housing 5 which is not connected to the cord 6. However, two cords may be respectively connected to the left and right housings 5 such that sound signals are respectively supplied to the sound processing circuits in the left and right housings 5.

The ear pads 7 are elastic and are provided on the surfaces facing the temporal region of the user in the housings 5. The ear pads 7 are interposed between the housings 5 and the temporal region of the user and thereby function as buffer members between the housings 5 and the temporal region of the user. In other words, when the headphones 1 are worn, the ear pads 7 prevent the housings 5 formed using rigid materials which are difficult to deform, from giving a sense of discomfort or pain to the user by coming into direct contact with the temporal region of the user.

The ear pads 7 also improve sound quality such as improvement in reproducibility of bass by sealing the spaces (hereinafter, referred to as “ear pad inner spaces”) formed by the ear pads 7 and the temporal region of the user. In addition, the ear pads 7 prevent sound output from the speakers from being leaked to the outside. The ear pads 7 blocks noise from the outside and make it easy to listening to sounds from the spealers.

Guard nets 8 are provided between the ear pads 7 and the housings 5. The guard nets 8 prevent dust from entering the speaker and the sound processing circuits contained in the housings 5.

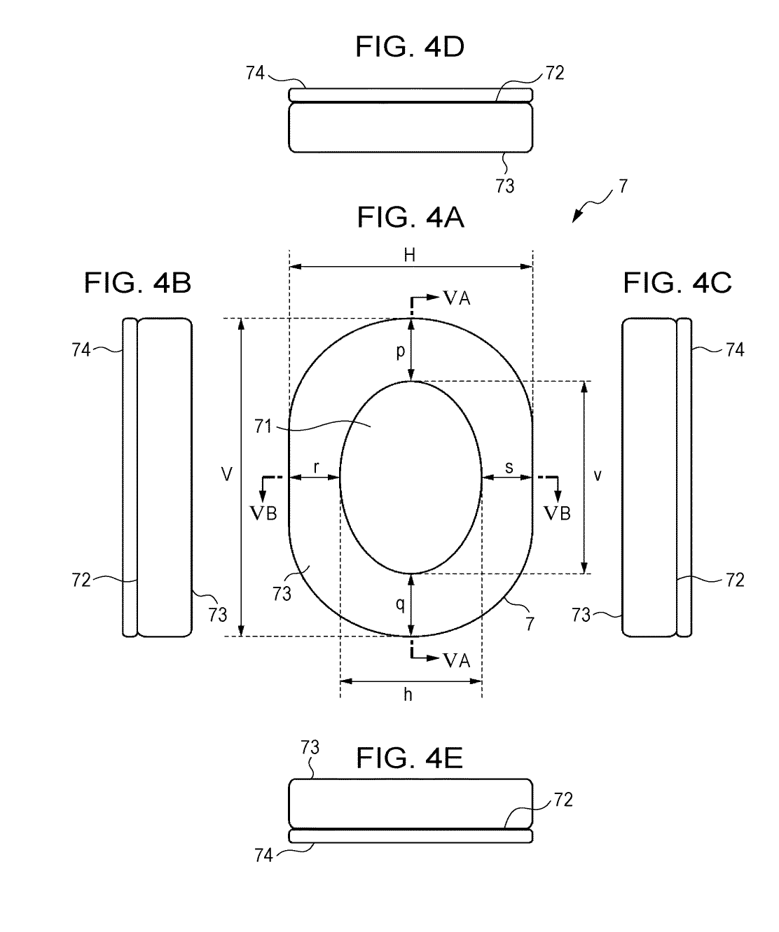

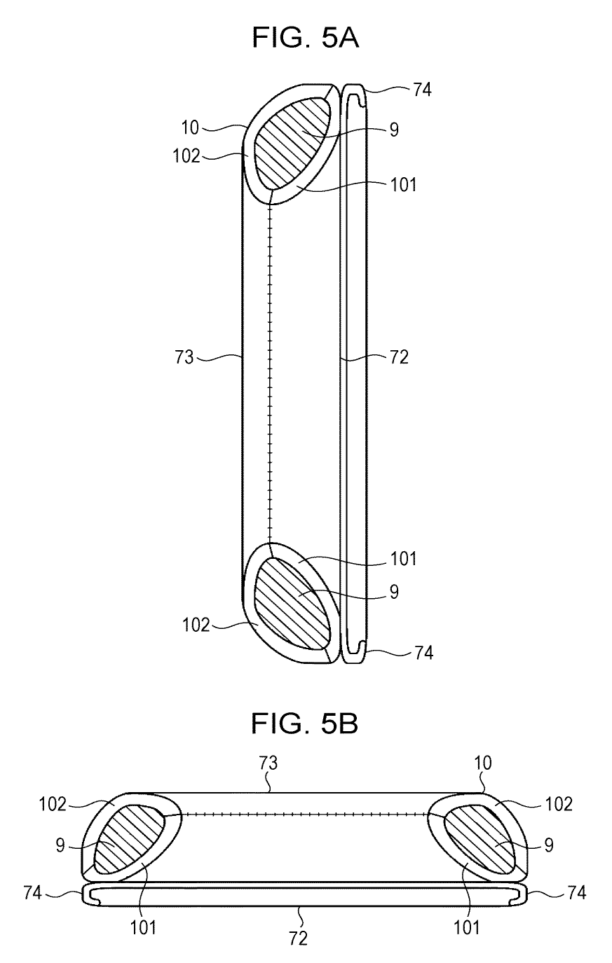

FIGS. 4A to 4E are exterior views of the ear pad 7. FIG. 4A is a front view (a view seen from the side coming into contact with the temporal region of a user), FIGS. 4B and 4C are side views, FIG. 4D is a plan view, and FIG. 4E is a bottom view. In addition, FIGS. 5A and 5B are cross-sectional views of the ear pad 7, in which FIG. 5A is a cross-sectional view taken along the line VA-VA in FIG. 4A, and FIG. 5B is a cross-sectional view taken along the line VB-VB in FIG. 4A.

The ear pad 7 has an opening portion 71 for containing the ear of a user at the center, and is formed in a vertically long ring shape when seen from the front side. The ear pad 7 has a bottom surface 72 positioned at the housing 5 side in a state of being installed in the housing 5, a contact surface 73 coming into contact with the temporal region of the user when the headphones 1 are worn, and a connection portion 74 connected to the housing 5.

The ear pad 7 is formed such that a difference between the outer size V and the inner size (the diameter of the opening portion 71 in the vertical direction) v increases, that is, the width of the contact surface 73 increases, in the vertical direction. On the other hand, the ear pad 7 is formed such that a difference between the outer size H and the inner size (the diameter of the opening portion 71 in the horizontal direction) h decreases, that is, the width of the contact surface 73 decreases, in the horizontal direction.

In addition, in the vertical direction, the distance between the outer circumferential upper end and the inner circumferential upper end (the vertical width p of the contact surface 73) is formed so as to be substantially the same as the distance between the outer circumferential lower end and the inner circumferential lower end (the vertical width q of the contact surface 73). In the horizontal direction, the distance between the outer circumference left end and the inner circumference left end (the horizontal width r of the contact surface 73) is formed so as to be substantially the same as the distance between the outer circumference right end and the inner circumference right end (the horizontal width s of the contact surface 73).

In this way, in the ear pad 7 according to the embodiment of the present disclosure, the vertical widths p and q of the contact surface 73 are formed to be large, and the horizontal widths r and s of the contact surface 73 are formed to be small. The opening portion 71 of the ear pad 7 may be formed so as to satisfy the above conditions and have an appropriate size capable of containing the ear of a user without generation of an excess gap.

The ear pad 7 includes a buffer portion 9 having a ring shape and a cover 10 covering the buffer portion 9. The buffer portion 9 is formed using an elastic material such as, for example, urethane foam, cotton, or chemical fiber. However, the material forming the buffer portion 9 is not limited thereto, and may be any material as long as it has appropriate elasticity.

The cover 10 covers the entire surface of the buffer portion 9 and comes into direct contact with the temporal region of a user when the headphones 1 are worn. In the first embodiment, the cover 10 includes a bottom surface side covering portion 101 covering the bottom surface 72 in the buffer portion 9 and a contact surface side covering portion 102 covering the contact surface 73 in the buffer portion 9. The bottom surface side covering portion 101 and the contact surface side covering portion 102 are coupled to each other by, for example, stitching. The cover 10 is preferably formed using a material which feels comfortable, such as, for example, natural leather or synthetic leather.

The connection portion 74 is provided on the outer circumference of the ear pad 7 in the housing 5 side and is formed in a substantially U shape in a cross-section, which is open in the central direction of the ear pad 7. The connection portion 74 extends toward the housing 5 and is formed such that the front end thereof faces the central direction of the ear pad 7. The part which extends in the central direction of the ear pad 7 is inserted into a groove (not shown) for ear pad installation formed on the outer circumference of the housing 5, and thereby the ear pad 7 is fixed to the housing 5. The ear pad 7 can be removed from the housing 5 and exchanged.

### 1.2. Effects Achieved by Ear Pad

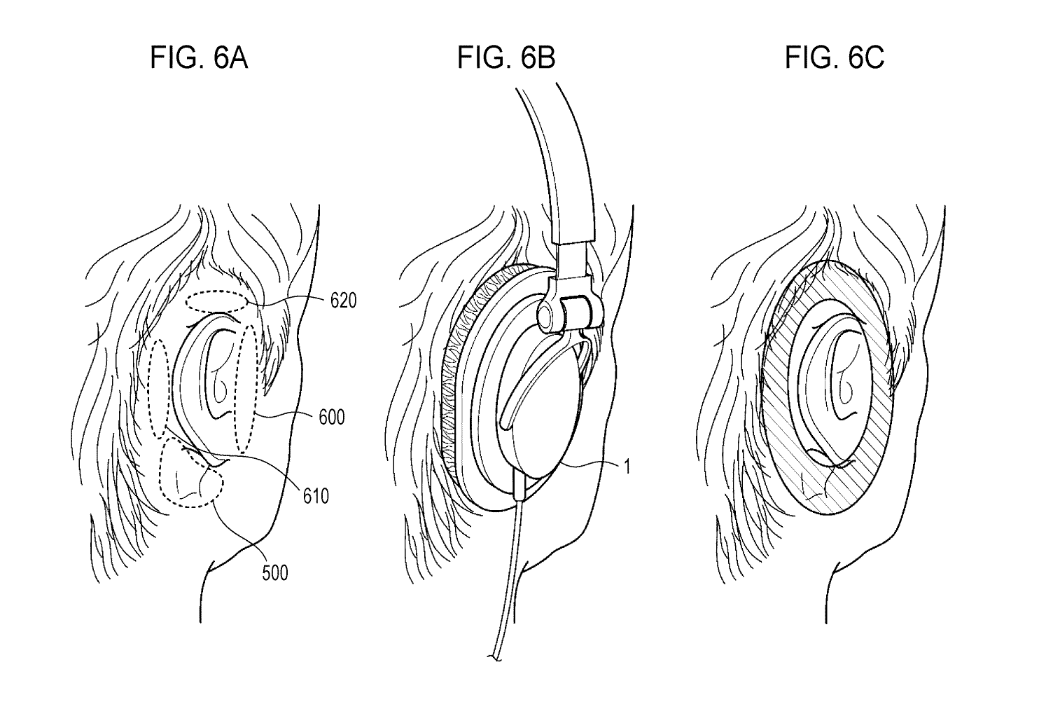

The ear pad 7 according to the first embodiment has the configuration described above. Next, effects achieved by the ear pad 7 will be described. As shown in FIG. 6A, there is an uneven region 500 where unevenness caused by the upper end of the jaw, the neck, and the like, exists behind and under the ear of a person. When the headphones 1 are worn, a gap between the unevenness in the uneven region 500 and the ear pad 7 is generated, and the ear pad inner space communicated with the outside, thereby reducing the sealing performance. If the sealing performance is reduced, sound leakage to the outside from the headphones 1, the penetration of noise from the outside, decrease in sound quality, and the like occur.

As shown in FIG. 6A, there is hair at the front side of the ear (the face direction in the state of wearing the headphones 1), in the rear side of the ear (the direction of the back of the head in the state of wearing the headphones 1), and in the upper side of the ear. When the headphones 1 are worn, if the ear pad 7 does not come into direct contact with the skin but the hair is interposed between the ear pad 7 and the skin of the temporal region, the ear pad inner space communicates with the outside via the hair portion, and thereby the sound leakage to the outside, the penetration of noise, and the like occur.

FIG. 6B shows a state where a user wears the headphones 1. In the state of wearing the headphones 1, as shown in FIG. 6C, the contact surface 73 of the ear pad 7 comes into contact with the vicinity of the ear in the temporal region. The range marked with the diagonal lines in FIG. 6C is a region where the contact surface 73 comes into contact therewith.

In the ear pad 7 according to the embodiment of the present disclosure, the width of the contact surface 73 is formed to be large in the vertical direction. Thereby, the contact area of the ear pad 7 with the skin increases when the ear pad 7 is deformed by being pressed by the headband 2 in the temporal region direction. Therefore, as shown in FIG. 6C, the ear pad 7 can cover the overall surface of the uneven region 500 which exists behind and under the ear. Therefore, it is possible to prevent the ear pad inner space from communicating with the outside due to the occurrence of a gap between the unevenness in the uneven region 500 and the ear pad 7, thereby increasing the sealing performance. Accordingly, the contact surface 73 is preferably formed such that the width in the vertical direction, particularly, the width of the lower part in the vertical direction can cover the overall surface of the uneven region 500.

The buffer portion 9 is formed using an elastic material such as, for example, urethane foam, cotton, or chemical fiber. Therefore, the ear pad 7 is deformed following the unevenness in the uneven region 500 when being pressed by the headband 2 in the temporal region direction. Thereby, it is possible to more reliably prevent the ear pad inner space from communicating with the outside due to the occurrence of a gap between the unevenness and the ear pad 7, thereby increasing the sealing performance.

The contact surface 73 of the ear pad 7 according to the embodiment of the present disclosure is formed to have a small width in the horizontal direction. Thereby, in the front side (the face direction) and the rear side (the direction of the back of the head) of the ear, the contact surface 73 of the ear pad 7 can come into direct contact with the hairless regions 600 and 610 which exist between the hair and the ear. In other words, it is possible to suppress the hair from being inserted by the ear pad 7. Therefore, it is possible to prevent the ear pad inner space from communicating with the outside in the hair portion, thereby increasing the sealing performance. The hairless regions indicate regions where hair does not grow around the ear in the temporal region, or even if hair grows, the amount thereof is very minute (including a case where only downy hair grows).

As described above, the contact surface 73 of the ear pad 7 is formed to be wide in the vertical direction, and an effect of increasing the sealing performance can be achieved. However, the effect of increasing the sealing performance is not necessarily proportional to the width of the ear pad 7.

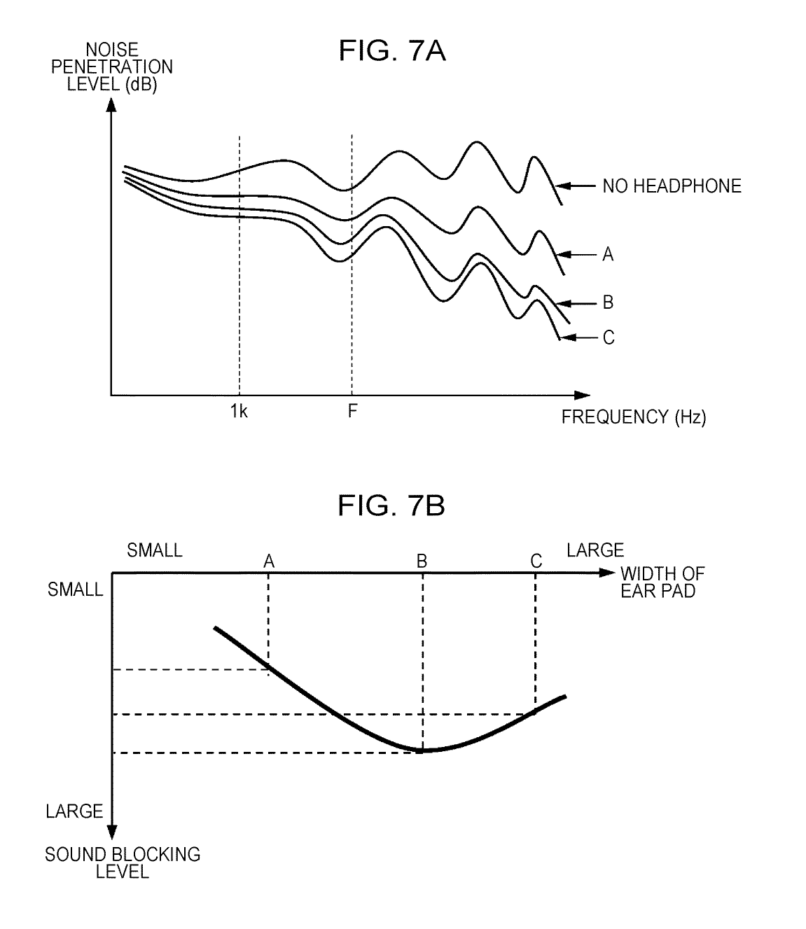

FIG. 7A shows a comparison result of the noise penetration levels of three ear pads of which inner sizes are the same as and outer sizes are different from each other, which thus have different widths of the contact surfaces in the vertical direction. The ear pad A has the smallest width, the ear pad B has the second largest width, and the ear pad C has the largest width.

As can be seen from the result shown in FIG. 7A, all of the ear pad A, the ear pad B, and the ear pad C can reduce the noise penetration level. However, the ear pad which reduces the noise penetration level most is not the ear pad C having the largest width in the vertical direction but the ear pad B. The noise penetration level of the ear pad C having the largest width of the contact surface in the vertical direction is larger than that of the ear pad B.

FIG. 7B shows a comparison result of the sound blocking levels for the ear pad A, the ear pad B, and the ear pad C at a predetermined frequency F shown in FIG. 7A. When the width of the contact surface of the ear pad in the vertical direction increases from the ear pad A to the ear pad B, the sound blocking level increases in proportion thereto. However, when the width of the contact surface in the vertical direction further increases from the ear pad B to the ear pad C, the sound blocking level decreases adversely. The width of the ear pad B gives the maximal sound blocking level, and if the width of the ear pad is larger than that of the ear pad B, the sound blocking level decreases adversely.

From the comparison result, it can be seen that further increase in the width of the contact surface of the ear pad in the vertical direction is not necessarily good, and there is an optimal value of the width of the ear pad for increasing to the greatest sealing performance. This may be because if the width of the contact surface increases too greatly, an amount of inserted hair increases, and thereby the ear pad fits loosely, or the lower end of the contact surface protrudes out from the jaw-line, and thereby the ear pad inner space communicates with the outside.

Considering this, the ear pad 7 according to the embodiment of the present disclosure is configured such that the outer size in the vertical direction (the diameter of the ear pad 7 in the vertical direction) indicated by V and the inner size in the vertical direction (the diameter of the opening portion 71 in the vertical direction) indicated by v in FIG. 4A satisfy the relationship expressed by the following Equation 1.

1.5≦(V/v)≦1.8 [Equation 1]

The comparison results shown in FIGS. 7A and 7B are also applied to the width of the contact surface in the horizontal direction. In other words, if the width decreases too greatly as compared with the optimal value of the width in the horizontal direction, the noise penetration level increases adversely, and even if the width increases as compared with the optimal value, the noise penetration level increases. Further reduction in the width of the contact surface of the ear pad in the horizontal direction is not necessarily good, and there is an optimal value of the width of the ear pad for increasing to the greatest sealing performance. Considering this, the ear pad 7 according to the embodiment of the present disclosure is configured such that the outer size in the horizontal direction of the ear pad 7(the diameter of the ear pad 7 in the horizontal direction) indicated by H and the inner size in the horizontal direction (the diameter of the opening portion 71 in the horizontal direction) indicated by h in FIG. 4A satisfy the relationship expressed by the following Equation 2. In addition, Equation 2 reflects therein the results of measurement tests actually performed for a plurality of subjects.

1.8≦(H/h)≦2.1 [Equation 2]

### 2. Second Embodiment

### 2.1. Configuration of Ear Pad

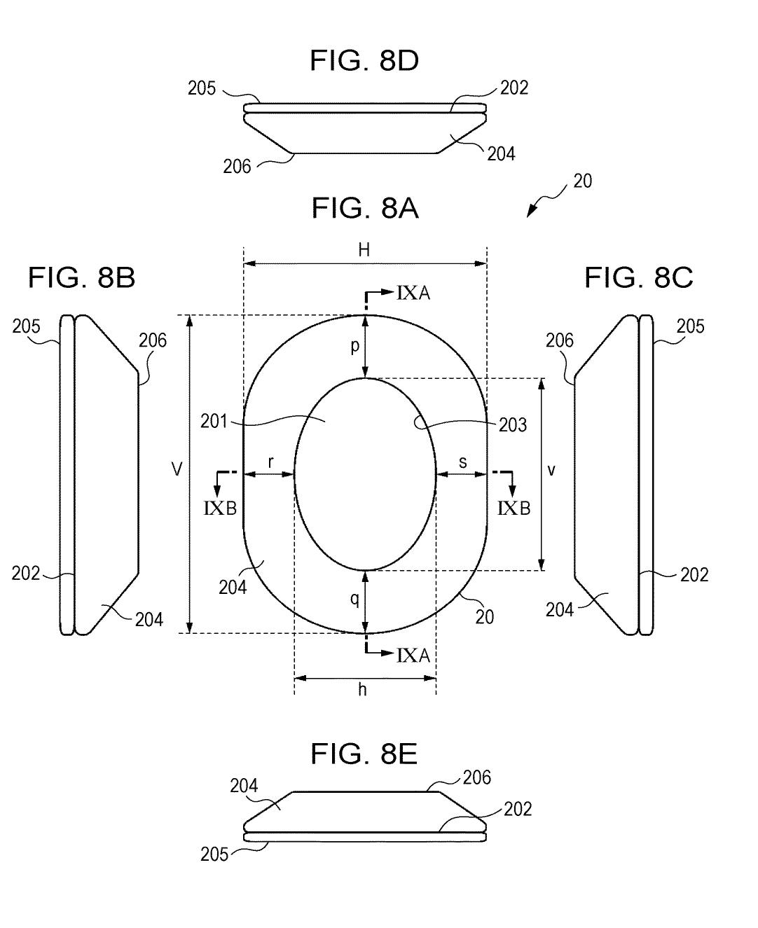

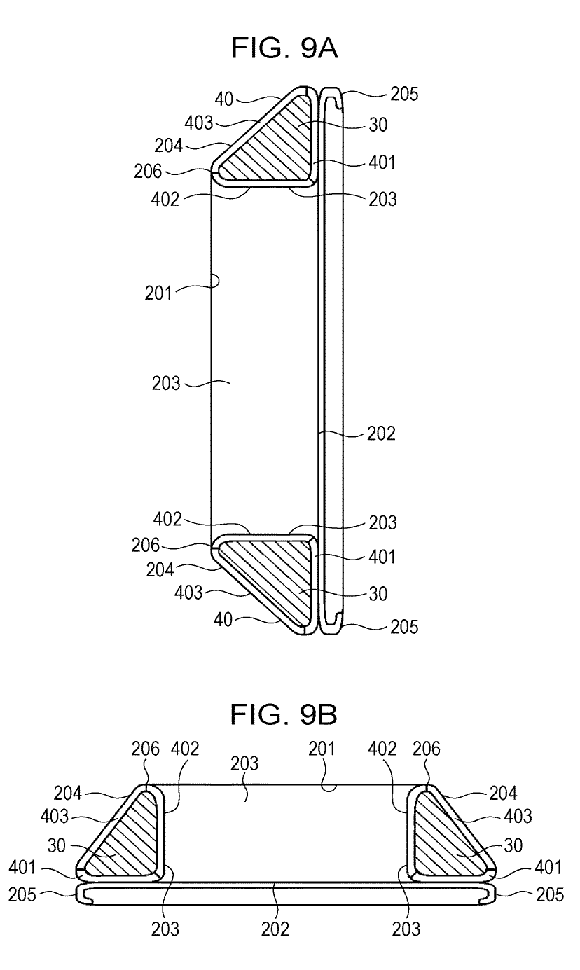

FIGS. 8A to 8E are exterior views of an ear pad 20 according to a second embodiment of the present disclosure. FIG. 8A is a front view (a view seen from the side coming into contact with the temporal region of a user), FIGS. 8B and 8C are side views, FIG. 8D is a plan view, and FIG. 8E is a bottom view. In addition, FIGS. 9A and 9B are cross-sectional views of the ear pad 20, in which FIG. 9A is a cross-sectional view taken along the line IXA-IXA in FIG. 8A, and FIG. 9B is a cross-sectional view taken along the line IXB-IXB in FIG. 8A. The configurations except for the ear pad 20 of the headphones 1 are the same as those in the first embodiment, and thus the description thereof will be omitted.

The ear pad 20 has an opening portion 201 for containing the ear of a user substantially at the center, and is formed in a vertically long ring shape when seen from the front side. The ear pad 20 has a bottom surface 202 positioned at the housing 5 side in a state of being installed in the housing 5, an inner circumference lateral surface 203 which is a lateral surface of the opening portion 201, a contact surface 204 coming into contact with the temporal region of the user when the headphones 1 are worn, and a connection portion 205 connected to the housing 5. In the same manner as the first embodiment, the ear pad 20 is formed such that a difference between the outer size and the inner size increases, that is, the width of the contact surface 204 of the ear pad 20 increases, in the vertical direction. In addition, the ear pad 20 is formed such that a difference between the outer size and the inner size decreases, that is, the width of the contact surface 204 decreases, in the horizontal direction.

In addition, in the same manner as the first embodiment, in the vertical direction, the distance between the outer circumferential upper end and the inner circumferential upper end (the vertical width p of the contact surface 204) is formed so as to be substantially the same as the distance between the outer circumferential lower end and the inner circumferential lower end (the vertical width q of the contact surface 204). In the horizontal direction, the distance between the outer circumference left end and the inner circumference left end (the horizontal width r of the contact surface 204) is formed so as to be substantially the same as the distance between the outer circumference right end and the inner circumference right end (the horizontal width s of the contact surface 204).

In the second embodiment, as shown in FIGS. 8B to 8E, the contact surface 204 is formed in a tilted shape so as to get narrower toward the opening portion 201. Therefore, the ear pad 20 is formed such that the width of the contact surface 204 decreases as the height from the bottom surface 202 increases.

In addition, the inner circumference lateral surface 203 is formed so as to substantially vertically stand from the bottom surface 202. The inner circumference lateral surface 203 and the contact surface 204 are coupled to each other at an inner circumferential upper edge 206 of the opening portion 201. Thereby, the contact surface 204 of the ear pad 20 is formed so as to have the greatest height from the bottom surface 202 at the inner circumferential upper edge 206 of the opening portion 201.

The ear pad 20 includes a buffer portion 30 having a ring shape and a cover 40 covering the buffer portion 30. The buffer portion 30 is formed using an elastic material such as, for example, urethane foam, cotton, or chemical fiber.

The cover 40 is made of natural leather, synthetic leather, or the like, covers the entire surface of the buffer portion 30, and comes into direct contact with the temporal region of the user. In the second embodiment, the cover 40 is constituted by a total of three covering portions, a first covering portion 401, a second covering portion 402, and a third covering portion 403. The first covering portion 401, the second covering portion 402, and the third covering portion 403 are coupled to each other by, for example, stitching.

The first covering portion 401 covers the bottom surface 202 side in the buffer portion 30. The second covering portion 402 covers the inner circumference lateral surface 203 side in the buffer portion 30. The second covering portion 402 is provided so as to substantially stand with respect to the bottom surface 202 and the first covering portion 401 through the coupling to one end of the first covering portion 401. The third covering portion 403 covers the contact surface 204 side in the buffer portion 30 through the coupling to the other end of the first covering portion 401 and the front end side of the second covering portion 402.

In this way, the cover 40 is formed using the three covering portions, the first covering portion 401, the second covering portion 402, and the third covering portion 403. Thereby, the first covering portion 401, the second covering portion 402, and the third covering portion 403 respectively form sides of a triangle, and can cover the buffer portion 30 having a substantial right triangle shape in a cross-section, along the shape thereof. Therefore, it is possible to easily implement the ear pad 20 having a substantial right triangle in a cross-section. However, the cover 40 is not necessarily formed of three sheets of leather. In a case where a substantial right triangle shape in a cross-section capable of achieving the effect according to the embodiment of the present disclosure is implemented using one or two sheets of leather, the cover 40 may be formed using one or two sheets of leather.

### 2.2. Effects Achieved by Ear Pad

The ear pad 20 according to the second embodiment has the configuration described above. Next, the effects achieved by the ear pad 20 will be described. As described in the first embodiment with reference to FIGS. 6A to 6C, there is the uneven region 500 where the unevenness such as the upper end of the chin exists behind and under the ear of a person. When the headphones 1 are worn, a gap between the unevenness in the uneven region 500 and the ear pad 20 is generated, and the ear pad inner space communicates with the outside, thereby reducing the sealing performance. Therefore, sound leakage to the outside from the headphones 1, the penetration of noise from the outside, and the like occur.

In addition, there is hair in the front side of the ear, in the rear side thereof, and in the upper side thereof. When the headphones 1 are worn, if the ear pad 20 does not come into direct contact with the skin but the hair is interposed between the ear pad 20 and the skin of the temporal region, the ear pad inner space communicates with the outside via the hair portion, and thereby the sound leakage to the outside, the penetration of noise, and the like occur.

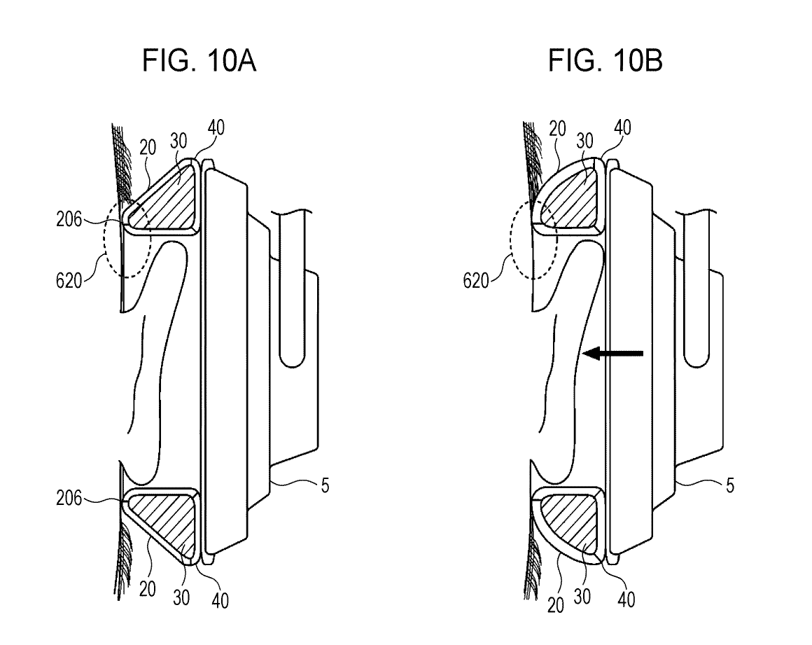

Therefore, in the second embodiment, the contact surface 204 of the ear pad 20 is formed in a tilted shape which is tilted toward the opening portion 201, and the contact surface 204 has the greatest height at the inner circumferential upper edge 206 of the opening portion 201. Thereby, the width of the inner circumferential upper edge 206 which is an upper edge of the contact surface 204 is small. Therefore, as shown in FIG. 10A, the inner circumferential upper edge 206 can more precisely come into contact with the hairless region 620 where hair does not grow between the ear and the hair that is present around the ear. This is also the same for the hairless regions 600 and 610, and the inner circumferential upper edge 206 can more precisely come into contact with the hairless regions 600 and 610.

As shown in FIG. 10B, even if the ear pad 20 is deformed by being pressed by the headband 2 and thus the hair is interposed between the ear pad 20 and the skin, since the inner circumferential upper edge 206 comes into direct contact with the skin, there is no case where the inner space of the ear pad 20 communicates with the outside via the hair portion. In other words, it is possible to heighten the sealing performance.

In the same manner as the first embodiment, in the second embodiment as well, the width of the contact surface 204 of the ear pad 20 is formed to be large in the vertical direction. Thereby, the contact area of the ear pad 20 with the skin increases when the ear pad 20 is deformed by being pressed by the headband 2 in the temporal region direction. Therefore, in the same manner as the first embodiment, as shown in FIG. 6C, the ear pad 20 can cover the overall surface of the uneven region 500 which is present behind and under the ear. Therefore, it is possible to prevent the ear pad inner space from communicating with the outside due to the occurrence of a gap between the unevenness in the uneven region 500 and the ear pad 20, thereby increasing the sealing performance.

The ear pad 20 in the second embodiment has the contact surface 204 which is formed in the tilted shape, and thus the width of the contact surface 204 decreases as the height from the bottom surface 202 increases. In other words, the width of the contact surface 204 becomes smaller so as to get narrower in a direction of coming into contact with the temporal region. Thereby, the ear pad 20 has increasing flexibility, and thus an amount of the ear pad to be deformed when being pressed by the headband 2 increases. Therefore, when being pressed by the headband 2, the ear pad 20 deforms its shape following the unevenness behind the ear, under the ear, and the like. Accordingly, it is possible to prevent a gap between the unevenness and the ear pad 20 from occurring, thereby increasing the sealing performance.

In addition, in the second embodiment, the contact surface 204 of the ear pad 20 is formed in the tilted shape, and thus the width of the contact surface 204 decreases as the height increases. Therefore, the inner circumferential upper edge 206 which is the front end of the contact surface 204 is deformed to be forced to enter the pits of the uneven region 500, and thus it is possible to more reliably prevent a gap between the ear pad 20 and the unevenness in the uneven region 500 from occurring.

### 3. Modified Examples

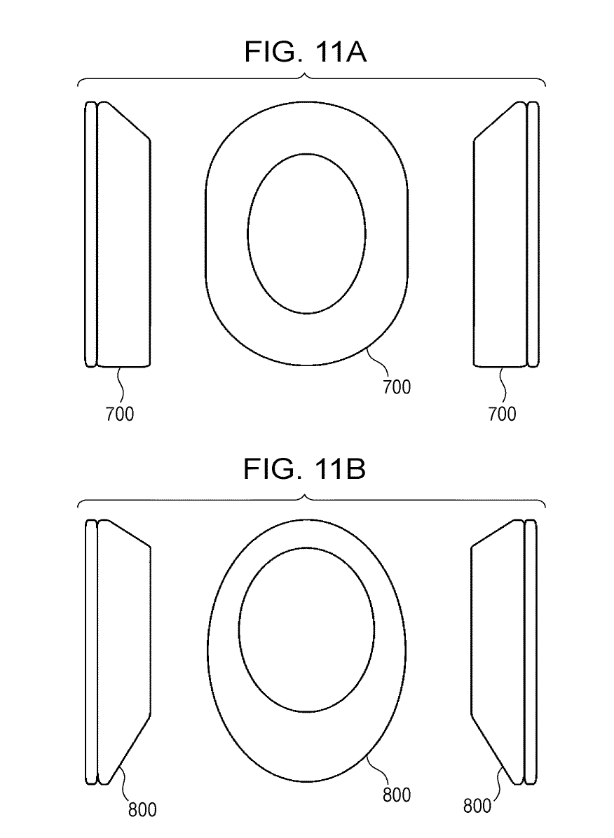

As above, although an embodiment of the present disclosure has been described in detail, the present disclosure is not limited to the above-described embodiment, and may have various kinds of modifications based on the technical spirit of the present disclosure. For example, the taper given to the contact surface of the ear pad is not necessarily given to the entire circumference of the ear pad, and only the upper part may be formed in a tilted shape in the vertical direction such as an ear pad 700 shown in FIG. 11A.

Such as in an ear pad 800 shown in FIG. 11B, the width of the contact surface at the upper part in the vertical direction may be formed to be small in the same manner as the width of the contact surface in the horizontal direction, and the width of the contact surface at the lower part in the vertical direction may be formed to be large. By this configuration, the front end of the ear pad can more precisely come into contact with the hairless region that is present in the upper side of the ear. Thereby, even if the ear pad is deformed by being pressed by the headband and thus the hair is interposed between the ear pad and the skin, since the front end of the ear pad comes into direct contact with the skin, there is no case where the inner space of the ear pad communicates with the outside via the hair portion. In other words, it is possible to heighten the sealing performance.

In addition, the ear pad according to the second embodiment is configured by combining the feature where the width of the contact surface in the vertical direction is large and the width of the contact surface in the horizontal direction is small with the feature where the contact surface is formed in the tilted shape. However, the two features are not necessarily combined and only one of the two features may be employed so as to configure the ear pad. The first embodiment employs the feature where the width in the vertical direction is large and the width horizontal direction is small. Unlike this, instead of employing the feature where the width in the vertical direction is large and the width horizontal direction is small, the ear pad may be configured by only the feature where the contact surface is formed in the tilted shape.

In the second embodiment, the example where the cross-section of the ear pad is substantially triangular as shown in FIGS. 9A and 9B has been described. However, the cross-section is not limited to the triangular shape. The cross-section may be formed in a substantially trapezoidal shape by the bottom surface, the inner circumference lateral surface substantially perpendicular to the bottom surface, the upper surface substantially parallel to the bottom surface, and the tilted surface. In this case, the upper surface and the tilted surface function as a contact surface. By this configuration as well, since the contact surface includes the tilted surface, and the width of the contact surface decreases according to the increase in height, it is possible to achieve the same effect as in the second embodiment.

In addition, in the above embodiments, the case where the ear pads according to the embodiment of the present disclosure are applied to the headphones having the headband which straddles the head is described. However, the ear pad according to the embodiment of the present disclosure is applicable to headphones of a type of installing the housing in the ear without using the headband which hangs the headphones on the ear.

In addition, in a case where the buffer portion 9 is made of a material having high air tightness, the cover is not necessarily formed.

The present disclosure contains subject matter related to that disclosed in Japanese Priority Patent Application JP 2010-196012 filed in the Japan Patent Office on Sep. 1, 2010, the entirety of which is hereby incorporated by reference.

It should be understood by those skilled in the art that various modifications, combinations, sub-combinations and alterations may occur depending on design requirements and other factors insofar as they are within the scope of the appended claims or the equivalents thereof.

What is claimed is:

1. An ear pad for use with a headphone apparatus, the ear pad comprising:
a pad portion comprising an outer circumference and an inner circumference, the inner circumference defining an opening;
wherein the pad portion comprises an upper width between the outer and inner circumferences along a vertical mid-line of the pad portion, a lower width between the outer and inner circumferences along the vertical mid-line of the pad portion, a left side width between the outer and inner circumferences along a horizontal mid-line of the pad portion, and a right side width between the outer and inner circumferences along the horizontal mid-line of the pad portion; and
wherein the lower width of the pad portion is greater than at least one of the left and right side widths of the pad portion.

2. The ear pad of claim 1, wherein:
the lower width of the pad portion is greater than each of the left and right side widths of the pad portion.

3. The ear pad of claim 1, wherein:
the upper width of the pad portion is greater than at least one of the left and right side widths of the pad portion.

4. The ear pad of claim 3, wherein:
the upper width of the pad portion is greater than each of the left and right side widths of the pad portion.

5. The ear pad of claim 1, wherein:
the upper width of the pad portion is the same as the lower width of the pad portion.

6. The ear pad of claim 1, wherein:
the left side width of the pad portion is the same as the right side width of the pad portion.

7. The ear pad of claim 1, wherein:
the vertical mid-line intersects the outer circumference at an outer upper point and an outer lower point and intersects the inner circumference at an inner upper point and an inner lower point;
V is the distance between the outer upper point and the outer lower point, and v is the distance between the inner upper point and the inner lower point; and
1.5≦(V/v)≦1.8.

8. The ear pad of claim 1, wherein:
the horizontal mid-line intersects the outer circumference at an outer left point and an outer right point and intersects the inner circumference at an inner left point and an inner right point;
H is the distance between the outer left point and the outer right point, and h is the distance between the inner left point and the inner right point; and
1.8≦(H/h)≦2.1.

9. The ear pad of claim 1, wherein:
the pad portion comprises a contact surface configured to contact a temporal region of a user and a bottom surface opposite the contact surface; and
at least a portion of the contact surface is tilted such that a height of the pad portion from the bottom surface to the contact surface along at least a portion of the inner circumference is greater than a height of the pad portion from the bottom surface to the contact surface along at least a portion of the outer circumference.

10. The ear pad of claim 1, wherein:
the pad portion comprises a contact surface configured to contact a temporal region of a user and a bottom surface opposite the contact surface; and
the vertical mid-line intersects the outer circumference at an outer point above the horizontal mid-line and intersects the inner circumference at an inner point above the horizontal mid-line; and
a height of the pad portion from the bottom surface to the contact surface at the inner point is greater than a height of the pad portion from the bottom surface to the contact surface at the outer point.

11. The ear pad of claim 1, wherein:
a cross-section of the pad portion along the upper width is substantially triangular.

12. The ear pad of claim 1, wherein:
at least a portion of the pad portion comprises a bottom surface, an inner circumference lateral surface substantially normal to the bottom surface, and a contact surface that is titled with respect to the bottom surface and the inner circumference lateral surface.

13. The ear pad of claim 1, wherein the pad portion comprises:
a buffer portion comprising an elastic material; and
a cover configured to cover the buffer portion.

14. The ear pad of claim 13, wherein:
the buffer portion comprises first, second and third sides that join to form a triangular cross-sectional shape; and
the cover comprises first, second and third covering portions respectively covering the first, second and third sides of the buffer portion.

15. The ear pad of claim 1, further comprising:
a connection portion coupled to a bottom surface of the pad portion, wherein the connection portion is configured to couple the pad portion to a groove associated with a housing of a headphone apparatus.

16. The ear pad of claim 15, wherein:
the connection portion has U-shaped cross-section.

17. A headphone apparatus comprising:
a headband comprising left and right ends;
left and right housings respectively coupled to the left and right ends of the headband; and
left and right ear pads respectively coupled to the left and right housings, each of the left and right ear pads comprising a pad portion having an outer circumference and an inner circumference, the inner circumference defining an opening;
wherein each pad portion comprises an upper width between the outer and inner circumferences along a vertical mid-line of the pad portion, a lower width between the outer and inner circumferences along the vertical mid-line of the pad portion, a left side width between the outer and inner circumferences along a horizontal mid-line of the pad portion, and a right side width between the outer and inner circumferences along the horizontal mid-line of the pad portion; and
wherein the lower width of each pad portion is greater than at least one of the left and right side widths of the pad portion.

18. A method of increasing the sealing performance of an ear pad comprising a pad portion, the pad portion comprising an upper width and a lower width along a vertical mid-line of the pad portion and a left side width and a right side width along a horizontal mid-line of the pad portion, wherein the lower width is greater than at least one of the left side width and the right side width, the method comprising:
positioning the pad portion so that a lower region of the pad portion including the lower width contacts a region under an ear of a user.

19. The method of claim 18, wherein the upper width is greater than at least one of the left side width and the right side width, the method further comprising:
positioning the pad portion so that an upper region of the pad portion including the upper width contacts a region above the ear of the user.

20. The method of claim 18, wherein at least a portion of a contact surface of the pad portion is tilted to form an edge along an inner circumference of an upper region of the pad portion including the upper width, the method comprising:
positioning the pad portion so that the edge contacts a region above the ear of the user.
